(19) United States
(12) Patent Application Publication
(10) Pub. No.: US 2007/0020329 A1
(43) Pub. Date: Jan. 25, 2007

(54) Methods of formulating linezolid

(76) Inventors: Ruth Tenengauzer, Raanana (IL); Minutza Leibovici, Netanya (IL); Ben-Zion Solomon, Petach Tikva (IL)

Correspondence Address: KENYON & KENYON LLP, ONE BROADWAY, NEW YORK, NY, 10004, US

(21) Appl. No.: 11/333,906

(22) Filed: Jan. 17, 2006

Related U.S. Application Data
(60) Provisional application No. 60/701,438, filed on Jul. 20, 2005.

(51) Int. Cl. A61K 9/20; A61K 31/5377

(52) U.S. Cl. 424/464; 514/235.2

(57) ABSTRACT

A method of formulating linezolid to provide a pharmaceutical composition comprising linezolid wherein the linezolid is linezolid Form IV substantially free of linezolid Form II, a solid pharmaceutical composition comprising linezolid Form IV substantially free of linezolid Form II and povidone, methods of treating a condition responsive to linezolid in a patient comprising administering to the patient a solid pharmaceutical composition comprising linezolid form IV substantially free of linezolid Form II, and methods of treating a condition responsive to linezolid in a patient comprising administering to the patient a solid pharmaceutical composition comprising linezolid form IV and povidone.

### CROSS REFERENCE TO RELATED APPLICATIONS

This application claims the benefit of U.S. provisional application No. 60/701,438, filed Jul. 20, 2005, the contents of which are expressly incorporated herein.

### FIELD OF THE INVENTION

The present invention is directed to a pharmaceutical composition containing linezolid form IV substantially free of linezolid form II.

The present invention is directed to a pharmaceutical composition containing linezolid form IV that does not substantially rearrange to linezolid form II.

The present invention is directed to a method of formulating linezolid to provide a pharmaceutical composition comprising linezolid wherein the linezolid is linezolid Form IV substantially free of linezolid Form II and methods of inhibiting the interconversion of linezolid Form IV into linezolid Form II wherein the method of manufacture is one that would normally occasion such interconversion, solid pharmaceutical compositions comprising linezolid Form IV substantially free of linezolid Form II and stable solid pharmaceutical compositions comprising linezolid Form IV that do not substantially convert into linezolid Form II over time, methods of treating a condition responsive to linezolid in a patient comprising administering to the patient a solid pharmaceutical composition comprising linezolid form IV substantially free of linezolid Form II, and methods of treating a condition responsive to linezolid in a patient comprising administering to the patient a solid pharmaceutical composition comprising linezolid form IV.

### BACKGROUND

Linezolid, chemically, N-[[(5S)-3-[3-fluoro-4-(4-morpholinyl)phenyl]-2-oxo-5-oxazolidinyl]methyl]acetamide has the chemical structure:

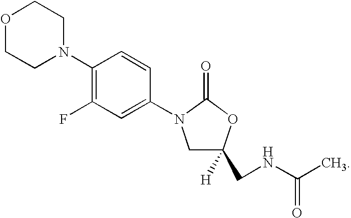

Linezolid is an anti bacterial agent.

Linezolid is known to exist in different crystal forms. Linezolid Form II is described in U.S. Pat. Nos. 6,444,813 and 6559305. Linezolid Form II is characterized by its X-Ray powder diffraction pattern and IR peaks. Linezolid Form II is characterized by an X-Ray powder diffraction pattern with the following peaks:

| 2-theta | relative intensity |
|---|---|
| 7.10 | 2 |
| 9.54 | 9 |
| 13.88 | 6 |
| 14.23 | 24 |
| 16.18 | 3 |
| 16.79 | 100 |
| 17.69 | 2 |
| 19.41 | 4 |
| 19.69 | 2 |
| 19.93 | 6 |
| 21.61 | 15 |
| 22.39 | 23 |
| 22.84 | 4 |
| 23.52 | 7 |
| 24.16 | 1 |
| 25.28 | 13 |
| 26.66 | 1 |
| 27.01 | 3 |
| 27.77 | 1 |

It is reported that linezolid Form II can be obtained by crystallization in a variety of solvents including water, ethyl acetate, methanol, ethanol, propanol, isopropanol, butanol, acetonitrile, acetone, methyl ethyl ketone, chloroform, toluene, and xylene.

Another Form of linezolid, designated linezolid Form IV is described in WO 2005/035530 (denominated form III) and is characterized by X-Ray powder diffraction pattern having peaks at 7.6, 9.6, 13.6, 14.9, 18.2, 18.9, 21.2, 22.3, 25.6, 26.9, 27.9, and 29.9 degrees 2-theta. Linezolid Form IV can be obtained, for instance, by heating Form II at a temperature above about 90° C., for a period of between 2 and 12 hours.

Citation of any reference in this section of this application is not to be construed that such reference is prior art to the present application.

### SUMMARY OF THE INVENTION

The invention relates to a pharmaceutical composition comprising polymorphically pure linezolid Form IV and a pharmaceutically acceptable excipient and to a method of making the pharmaceutical composition comprising polymorphically pure linezolid Form IV.

The invention also relates to a pharmaceutical composition comprising linezolid Form IV substantially free of linezolid form II and to a process for making the pharmaceutical composition comprising linezolid Form IV substantially free of linezolid form II.

The invention also relates to a pharmaceutical composition comprising polymorphically stable linezolid Form IV and a pharmaceutically acceptable excipient and to a method for making the pharmaceutical composition comprising polymorphically stable linezolid Form IV.

Definitions of the terms “polymorphically pure,” polymorphically stable,” and “substantially free” are provided below. As defined below the stability of polymorphically stable linezolid Form IV may be defined in terms of stability at 25° C. and 60% relative humidity over a period of time or at 40° C. and 75% relative humidity over a period of time. Preferably stability is defined with respect to 40° C. and 75% relative humidity over a period of time, preferably 3 months. It is preferred that the polymorphically stable linezolid is polymorphically pure linezolid for IV. Preferably, the polymorphically pure linezolid Form IV is substantially free, i.e., contains less than 20%, of other polymorphic forms of linezolid, more preferably less than 20% of linezolid Form II. All further embodiments of the invention are described with reference to linezolid Form IV substantially free of linezolid Form II, but are equally applicable to the broader definition.

In one embodiment, the pharmaceutical composition is solid. In one embodiment, the pharmaceutical composition is in the form of a tablet. In one embodiment, the pharmaceutical composition is in the form of a capsule.

In one embodiment, the pharmaceutical composition comprises povidone.

In one embodiment, the pharmaceutical composition is substantially free of sugar alcohols

In one embodiment, the pharmaceutical composition is substantially free of mannitol.

In one embodiment, the pharmaceutical compositions are prepared by wet granulation. In another embodiment the pharmaceutical compositions are prepared by dry granulation. In another embodiment the pharmaceutical compositions are prepared by direct compression.

In one embodiment, the invention is directed to a method of preparing a pharmaceutical composition comprising linezolid Form IV substantially free of linezolid Form II comprising wet granulating linezolid Form IV substantially free of linezolid Form II with a pharmaceutically acceptable excipient using a granulation solvent selected from the group consisting of water, isopropanol, and ethanol in the presence of povidone.

In one embodiment, the method is directed to a method of preparing a pharmaceutical composition comprising linezolid Form IV substantially free of linezolid Form II comprising wet granulating Form IV substantially free of linezolid Form II with an excipient that limits the amount of linezolid Form IV that is converted to linezolid Form II. In one embodiment, the excipient that limits the amount of linezolid Form IV that is converted to linezolid Form II is povidone. In one embodiment, the invention is directed to a method of preparing a pharmaceutical composition comprising linezolid Form IV substantially free of linezolid Form II comprising wet granulating linezolid Form IV with an excipient that limits the amount of linezolid Form IV that is converted to linezolid Form II and at least one other excipient. In one embodiment, the excipient that limits the amount of linezolid Form IV that is converted to linezolid Form II is povidone.

In one embodiment, the pharmaceutical compositions are prepared by dry granulation. In one embodiment, the invention is directed to a method of preparing a pharmaceutical composition comprising linezolid Form IV substantially free of linezolid Form II by dry granulating linezolid Form IV with an excipient that limits the amount of linezolid Form IV that is converted to linezolid Form II. In one embodiment, the excipient that limits the amount of linezolid Form IV that is converted to linezolid Form II is povidone. In one embodiment, the invention is directed to a method of preparing a pharmaceutical composition comprising linezolid Form IV substantially free of linezolid Form II comprising dry granulating linezolid Form IV with an excipient that limits the amount of linezolid Form IV that is converted to linezolid Form II and at least one other excipient. In one embodiment, the excipient that limits the amount of linezolid Form IV that is converted to linezolid Form II is povidone.

In another embodiment, the pharmaceutical compositions are prepared by direct compression. In one embodiment, the invention is directed to a method of preparing a pharmaceutical composition comprising linezolid Form IV substantially free of linezolid Form II comprising admixing linezolid Form IV substantially free of linezolid Form II with a pharmaceutically acceptable excipient to provide a mixture and direct compressing the mixture. In one embodiment, the excipient is an excipient limits the amount of linezolid Form IV that is converted to linezolid Form II. In one embodiment, the excipient that limits the amount of linezolid Form IV that is converted to linezolid Form II is povidone. In one embodiment, the invention is directed to a method of preparing a pharmaceutical composition comprising linezolid Form IV substantially free of linezolid Form II comprising admixing linezolid Form IV substantially free of linezolid Form II with a pharmaceutically acceptable excipient that limits the amount of linezolid Form IV that is converted to linezolid Form II and at least one other excipient to provide a mixture and direct compressing the mixture. In one embodiment, the excipient that limits the amount of linezolid Form IV that is converted to linezolid Form II is povidone.

The invention further relates to a method of treating a condition responsive to linezolid in a patient comprising administering to the patient a solid pharmaceutical composition comprising linezolid form IV substantially free of linezolid Form II. In one embodiment, the solid pharmaceutical composition comprising linezolid form IV substantially free of linezolid Form II is prepared according to the method of the invention. In one embodiment, the condition responsive to linezolid is a bacterial infection.

The invention further relates to a method of treating a condition responsive to linezolid in a patient comprising administering to the patient a solid pharmaceutical composition comprising linezolid form IV and povidone. In one embodiment, the condition responsive to linezolid is a bacterial infection.

### BRIEF DESCRIPTION OF THE DRAWINGS

FIG. 1 is an XRD diffractogram of linezolid Form IV.

FIG. 2 is an XRD diffractogram of linezolid Form II.

FIG. 3 is an XRD diffractogram of the linezolid pharmaceutical composition prepared in Experiment No. 1.

FIG. 4 is an XRD diffractogram of the linezolid pharmaceutical composition prepared in Experiment No. 2.

FIG. 5 is an XRD diffractogram of the linezolid pharmaceutical composition prepared in Experiment No. 3.

FIG. 6 is an XRD diffractogram of the linezolid pharmaceutical composition prepared in Experiment No. 4.

FIG. 7 is an XRD diffractogram of the linezolid pharmaceutical composition prepared in Experiment No. 5.

FIG. 8 is an XRD diffractogram of the linezolid pharmaceutical composition prepared in Experiment No. 6.

FIG. 9 is an XRD diffractogram of the linezolid pharmaceutical composition prepared in Experiment No. 7.

FIG. 10 is an XRD diffractogram of the linezolid pharmaceutical composition prepared in Experiment No. 8.

FIG. 11 is an XRD diffractogram of the linezolid pharmaceutical composition prepared in Experiment No. 9.

FIG. 12 is an XRD diffractogram of the linezolid pharmaceutical composition prepared in Experiment No. 10.

FIG. 13 is an XRD diffractogram of the linezolid pharmaceutical composition prepared in Experiment No. 11.

FIG. 14 is an XRD diffractogram of the linezolid pharmaceutical composition prepared in Experiment No. 12.

FIG. 15 is an XRD diffractogram of the linezolid pharmaceutical composition prepared in Experiment No. 13.

FIG. 16 is an XRD diffractogram of the linezolid pharmaceutical composition prepared in Experiment No. 14.

FIG. 17 is an XRD diffractogram of the linezolid pharmaceutical composition prepared in Experiment No. 15.

FIG. 18 is an XRD diffractogram of the linezolid pharmaceutical composition prepared in Experiment No. 16.

FIG. 19 is an XRD diffractogram of the linezolid pharmaceutical composition prepared in Experiment No. 17.

FIG. 20 is an XRD diffractogram of the linezolid pharmaceutical composition prepared in Experiment No. 18.

### DETAILED DESCRIPTION OF THE INVENTION

The invention is directed to a method of preparing a solid pharmaceutical composition comprising linezolid Form IV substantially free of linezolid Form II.

The phrase “linezolid Form IV,” as used herein means the polymorphic form of linezolid disclosed in WO2005/035530 (and refered to in WO2005/035530 as form III), the contents of which are herein incorporated in their entirety. WO2005/035530 describes how this form of linezolid may be prepared for use in accordance with the present invention.

The phrase “solid dosage form” or “solid pharmaceutical composition,” as used herein, means a dosage form that is a solid, i.e., not a liquid, and includes, but is not limited to, tablets, capsules, sugar coated tablets, film coated tablets, enteric coated tablets, multiple compressed tablets, controlled release tablets, effervescent tablets, suppositories, and buccal and sublingual tablets (See, Remington The Science and Practice of Pharmacy 20^{th} ed. (“Remington”), edited by A. Gennaro, Philadelphia College of Pharmacy and Science 2000 (the contents of which are expressly incorporated herein by reference hereto), p. 858-856). The term “solid dosage form,” as used herein, also includes suspensions of linezolid Form IV. The term “suspension,” as used herein means a dispersion containing finely divided insoluble material suspended in a liquid medium (See, Remington, p. 317).

The phrase “excipient” or pharmaceutically acceptable excipient,” as used herein, means an ingredient in a pharmaceutical composition other than the active ingredient (See, Remington, page 860). Excipients include, but are not limited to, diluents (inert substances to increase the bulk of the pharmaceutical composition), binders (agents used to impart cohesive qualities to a powdered material), lubricants (agents that prevent adhesion of material to a die or punch, reduce inter-particle friction, facilitate ejection of a tablet from a die cavity, and/or improve the rate of flow of a powder mixture), glidants (agents that improve the flow characteristics of a powder), disintegrants (agents that facilitate the breakup or disintegration of a tablet after administration), coloring agents (agents that impart a color to a dosage form), and flavoring agents (agents that impart a flavor to a dosage form) (See, Remington, page 860-863). Suitable excipients include, but are not limited to, those described in Remington (See, Remington, page 860-863).

The term “povidone,” as used herein means polyvinylpyrrolidone.

The phrase “substantially free of,” as used herein, means not more than 20%. Accordingly, the phrase “linezolid Form IV substantially free of linezolid Form II” means linezolid Form IV containing not more than 20% of linezolid Form II. In one embodiment, the linezolid Form IV contains not more than 15% of linezolid Form II. In one embodiment, the linezolid Form IV contains not more than about 10% of linezolid Form II. In one embodiment, the linezolid Form IV contains not more than about 5% of linezolid Form II.

The phrase “polymorphically pure compound,” as that term is used herein, means a polymorph of a compound that is substantially free of other polymorphs of the compound and the amorphous compound. Accordingly, the phrase “polymorphically pure linezolid Form IV” means linezolid Form IV that is substantially free of other polymorphs of linezolid and amorphous linezolid. In one embodiment, the polymorphically pure linezolid Form IV contains not more than 20% of other polymorphs of linezolid and amorphous linezolid. In one embodiment, the polymorphically pure linezolid Form IV contains not more than 15% of other polymorphs of linezolid and amorphous linezolid. In one embodiment, the polymorphically pure linezolid Form IV contains not more than 10% of other polymorphs of linezolid and amorphous linezolid. In one embodiment, the polymorphically pure linezolid Form IV contains not more than 5% of other polymorphs of linezolid and amorphous linezolid.

The phrase “polymorphically stable linezolid form IV,” as used herein, means linezolid form IV that shows not more than 10% conversion of linezolid Form IV to linezolid Form II when stored at 25° C./60% RH for 3 months. In one embodiment, the linezolid form IV shows not more than 10% conversion of linezolid Form IV to linezolid Form II when stored at 25° C./60% RH for 6 months. In one embodiment, the linezolid form IV shows not more than 30% conversion of linezolid Form IV to linezolid Form II when stored at 40° C./75% RH for 3 months. In one embodiment, the linezolid form IV shows not more than 25% conversion of linezolid Form IV to linezolid Form II when stored at 40° C./75% RH for 3 months. In one embodiment, the linezolid form IV shows not more than 20% conversion of linezolid Form IV to linezolid Form II when stored at 40° C./75% RH for 3 months.

The phrase “wet granulation,” as used herein, means a method of manufacturing a tablet that involves adding a binder to a mixture of the active ingredient and other excipients as a solution of the binder, and is well known to one of ordinary skill in the art (See, Remington, p. 865-868). Typically, wet granulation involves the steps of blending an active ingredient, in this case linezolid, with one or more solid excipients such as diluents and disintegrants to provide a powdered mix; wetting the powdered mix with a granulation solvent or a solution of a binding agent in a granulation solvent to provide a damp mass; screening the damp mass; drying the damp mass to provide a dried mass; screening the dried mass to provide granules; and forming the granules into a solid dosage form such as a capsule or tablet (See, Remington, page 865-868).

The phrase “dry granulation,” as used herein, means a method of manufacturing a tablet that avoids the use of a granulation solvent and the step of drying, as is required by wet granulation, and is well known to one of ordinary skill in the art (See, Remington, page 869). Typically, dry granulation involves the steps of weighing, mixing, slugging or compressing, dry screening, lubrication, and compression (See, Remington, page 869).

The phrase “direct compression,” as used herein, means a method of manufacturing a tablet by compressing tablets directly from powdered material without modifying the nature of the material itself, and is well known to one of ordinary skill in the art (See, Remington, page 869-870).

In one embodiment, the pharmaceutical composition is in the form of a tablet.

In one embodiment, the pharmaceutical composition is in the form of a capsule.

In one embodiment, the process involves the step of wet granulating linezolid Form IV substantially free of linezolid Form II with a pharmaceutically acceptable excipient using a grahulation solvent selected from the group consisting of water, isopropanol, ethanol in the presence of povidone, and water admixed with isopropanol.

In one embodiment, the granulating solvent is substantially free of ethanol.

In one embodiment, the solvent comprises water. In one embodiment, the solvent is water substantially free of a second solvent. In one embodiment, the solvent is water.

In one embodiment, the solvent is ethanol in the presence of povidone.

In one embodiment, the solvent is water admixed with isopropanol.

In one embodiment, the solvent comprises isopropanol. In one embodiment, the solvent is isopropanol substantially free of a second solvent. In one embodiment, the solvent is isopropanol.

In one embodiment, the linezolid Form IV substantially free of linezolid Form II is wet granulated with more than one pharmaceutically acceptable excipient.

In one embodiment, povidone is a pharmaceutically acceptable excipient.

Water admixed with ethanol or isopropanol means a mixture of water and ethanol or isopropanol wherein the mixture contains greater than about 25% water by weight, preferably greater than about 35% water by weight, more preferably greater than about 40% water by weight, and most preferably greater than about 50% water by weight.

It has been observed that preparing a pharmaceutical composition comprising linezolid Form IV and a pharmaceutically acceptable excipient by wet granulating the linezolid Form IV and a pharmaceutically acceptable excipient using ethanol (not in the presence of povidone) as the granulation solvent, a common granulation solvent, the resulting pharmaceutical composition contains substantial amounts of linezolid Form II or transforms completely to form II.

By wet granulating the linezolid Form IV and pharmaceutically acceptable excipient with water, isopropanol, or ethanol with the presence of povidone to form the pharmaceutical composition, the conversion of linezolid Form IV to linezolid Form II is limited or even eliminated. The term “limited,” as used herein, means that the amount of linezolid Form IV converted to linezolid Form II is less than the amount of linezolid Form IV that would be converted to linezolid Form II if a pharmaceutical composition was prepared by wet granulating the linezolid Form IV and pharmaceutically acceptable excipient with ethanol (not in the presence of povidone) as the granulation solvent.

The invention further relates to a method of preparing a solid pharmaceutical composition comprising linezolid Form IV substantially free of linezolid Form II wherein the method comprises wet granulating linezolid Form IV substantially free of linezolid Form II with a pharmaceutically acceptable excipient using a granulation solvent that is substantially free of ethanol.

In one embodiment, povidone is a pharmaceutically acceptable excipient.

Without wishing to be bound by theory, applicants suggest that linezolid Form IV is not more thermodynamically stable than linezolid Form II at room temperature. Although solid linezolid Form IV is kinetically stable at room temperature, i.e., it does not convert to linezolid Form II (over a time period of at least 3 months at temperatures between 25 and 40° C.), we have observed that when linezolid Form IV is contacted with solvents, in particular ethanol, it readily transforms to linezolid Form II, i.e., the thermodynamically more stable form of linezolid at room temperature. As a consequence, pharmaceutical compositions comprising linezolid Form IV substantially free of linezolid Form II are difficult to prepare.

In one embodiment, the invention relates to means a method of manufacturing a pharmaceutical composition of linezolid form IV substantially free of linezolid form II, that avoids the use of a liquid during formulation.

It has also been observed that the conversion of linezolid Form IV to linezolid Form II can be limited by dry granulating linezolid Form IV with a pharmaceutically acceptable excipient. Accordingly, the invention further relates to a method of preparing a solid pharmaceutical composition comprising linezolid Form IV substantially free of linezolid Form II wherein the method comprises dry granulating linezolid Form IV substantially free of linezolid Form II with a pharmaceutically acceptable excipient.

In one embodiment, the linezolid Form IV substantially free of linezolid Form II is dry granulated with more than one excipients.

In one embodiment, povidone is an excipient.

It has also been observed that the conversion of linezolid Form IV to linezolid Form II can be limited by direct compression of linezolid Form IV with a pharmaceutically acceptable excipient. Accordingly, the invention further relates to a method of preparing a solid pharmaceutical composition comprising linezolid Form IV substantially free of linezolid Form II wherein the method comprises admixing linezolid Form IV substantially free of linezolid Form II with a pharmaceutically acceptable excipient to provide a mixture and direct compressing the mixture.

In one embodiment, the linezolid Form IV substantially free of linezolid Form II is admixed with more than one pharmaceutically acceptable excipient to provide a mixture and the mixture is then direct compressed.

In one embodiment, povidone is an excipient.

It has also been observed that specific excipients, called “excipients that preserve Form IV linezolid” limit the conversion of linezolid Form IV to linezolid Form II, even when the pharmaceutical composition is prepared by wet granulation with ethanol as the granulation solvent. Accordingly, the invention further relates to a method of preparing a solid pharmaceutical composition comprising linezolid Form IV substantially free of linezolid Form II wherein the method comprises admixing linezolid Form IV substantially free of linezolid Form II with an excipient that preserves Form IV linezolid. The term “limit,” as used herein, means that the amount of linezolid Form IV converted to linezolid Form II is less than the amount of linezolid Form IV that would be converted to linezolid Form II if a pharmaceutical composition was prepared by in the absence of excipients that preserve Form IV linezolid.

In one embodiment, linezolid Form IV substantially free of linezolid Form II is admixed with an excipient that preserves Form IV linezolid and at least one other excipient to provide a mixture.

In one embodiment, the invention relates to a method of preparing a solid pharmaceutical composition comprising linezolid Form IV substantially free of linezolid Form II wherein the method comprises admixing linezolid Form IV substantially free of linezolid Form II with an excipient that preserves Form IV linezolid to provide a mixture and wet granulating the mixture using a solvent comprising ethanol as the granulating solvent. In one embodiment, the granulating solvent is ethanol.

An example of an excipient that preserves Form IV linezolid is povidone. Accordingly, the invention further relates to a method of preparing a solid pharmaceutical composition comprising linezolid Form IV substantially free of linezolid Form II wherein the method comprises admixing linezolid Form IV substantially free of linezolid Form II with povidone.

In one embodiment, the method involves admixing linezolid Form IV substantially free of linezolid Form II, povidone, and at least one other pharmaceutically acceptable excipient.

In one embodiment, the linezolid Form IV substantially free of linezolid Form II is admixed with povidone by wet granulating the linezolid Form IV substantially free of linezolid Form II and povidone. In one embodiment, the granulating solvent is ethanol. In one embodiment, the granulating solvent is ethanol admixed with water. In one embodiment, the granulating solvent is isopropanol. In one embodiment, the granulating solvent is isopropanol admixed with water. In one embodiment, the granulating solvent is water.

In one embodiment, linezolid Form IV substantially free of linezolid Form II, povidone, and at least one other pharmaceutically acceptable excipient are admixed by wet granulation.

In one embodiment, the linezolid Form IV substantially free of linezolid Form II is admixed with povidone by dry granulation.

In one embodiment, the linezolid Form IV substantially free of linezolid Form II is admixed with povidone and at least other excipient by dry granulation.

In one embodiment, the pharmaceutical composition is obtained by admixing the linezolid Form IV substantially free of linezolid Form II and povidone to provide a mixture and direct compressing the mixture.

In one embodiment, the linezolid Form IV substantially free of linezolid Form II is admixed with povidone and at least other excipient to provide a mixture and direct compressing the mixture.

The invention further relates to a solid pharmaceutical composition comprising linezolid Form IV substantially free of linezolid Form II.

The invention further relates to a solid pharmaceutical composition comprising linezolid Form IV substantially free of linezolid Form II and povidone.

In one embodiment, the pharmaceutical composition is a tablet. In one embodiment, the pharmaceutical composition is a capsule. The solid pharmaceutical compositions are made using methods well known to those skilled in the art (See, Remington, p. 858-893).

The invention further relates to a method of treating a condition responsive to linezolid in a patient comprising administering to the patient a solid pharmaceutical composition comprising linezolid form IV substantially free of linezolid Form II. In one embodiment, the solid pharmaceutical composition comprising linezolid form IV substantially free of linezolid Form II is prepared according to the method of the invention. In one embodiment, the condition responsive to linezolid is a bacterial infection.

The invention further relates to a method of treating a condition responsive to linezolid in a patient comprising administering to the patient a solid pharmaceutical composition comprising linezolid form IV and povidone. In one embodiment, the condition responsive to linezolid is a bacterial infection.

In each of the above-described embodiments of the invention, the composition is preferably substantially free of sugar alcohols such as mannitol.

### EXAMPLES

Various pharmaceutical compositions, in the form of a tablet, containing 300 mg of linezolid Form IV, were prepared by wet granulation, dry granulation, or direct compression.

### Examples 1-4

Method—Wet granulation.

Granulation Solution—Purified Water

Procedure:
1. Mix together the components of part I.
2. Add granulation solution (Purified Water) to the mix from step 1 to form a granulate.
3. Dry and mill the granulate from step 2.
4. Add components of part II and III to the milled granulate from step 3 and mix to get a final blend.

5. Compress the final blend into tablets.

TABLE 1

| | Ex- | Ex- | Ex- | |
|---|---|---|---|---|
| | ample 1 | ample 2 | ample 3 | Example 4 |
| Ingredients | mg/tab | mg/tab | mg/tab | mg/tab |
| Part I | | | | |
| Linezolid | 300.0 | 300.0 | 300.0 | 300.0 |
| Starch NF | 100.0 | 100.0 | 100.0 | 100.0 |
| Lactose Monohydrate NF | 100.0 | — | — | — |
| Mannitol | — | — | — | 100.0 |
| Cal. Phosphate Dibasic Anhyd. | — | 100.0 | 100.0 | — |
| USP | | | | |
| Povidone USP (PVP K-30) | — | — | 30.0 | — |
| Hydroxy Methyl Cellulose NF | 16.0 | 16.0 | — | 16.0 |
| Croscarmellose Sodium NF | — | — | 24.0 | — |
| Crospovidone USP | 24.0 | 24.0 | — | 24.0 |
| Part II | | | | |
| Lactose Spray Dried | 54.0 | — | — | 54.0 |
| Microcrystalline Cellulose NF | — | — | 40.0 | — |
| A-Tab (Cal. Phosphate Dibasic | — | 54.0 | — | — |
| Anhyd. USP) | | | | |
| Part III | | | | |
| Magnesium Stearate NF | 6.0 | 6.0 | 6.0 | 6.0 |
| Total | 600.0 | 600.0 | 600.0 | 600.0 |

### Examples 5-8

Method—Wet granulation

Granulation Solution—Ethanol

Procedure:
1. Mix together the components of part I.
2. Add granulation solution (Ethanol ) to the mix from step 1 to form a granulate.
3. Dry and mill the granulate from step 2.
4. Add components of part II and III to the milled granulate from step 3 and mix to get a final blend.

5. Compress the final blend into tablets.

TABLE 2

| | Ex- | Ex- | Ex- | |
|---|---|---|---|---|
| | ample 5 | ample 6 | ample 7 | Example 8 |
| Ingredients | mg/tab | mg/tab | mg/tab | mg/tab |
| Part I | | | | |
| Linezolid | 300.0 | 300.0 | 300.0 | 300.0 |
| Starch NF | 100.0 | 100.0 | 100.0 | 100.0 |
| Lactose Monohydrate NF | 100.0 | — | — | — |
| Cal. Phosphate Dibasic Anhyd. | — | 100.0 | 100.0 | — |
| USP | | | | |
| Mannitol | — | — | — | 100.0 |
| Povidone USP (PVP K-30) | — | — | 30.0 | — |
| Hydroxy Methyl Cellulose NF | 16.0 | 16.0 | — | 16.0 |
| Croscarmellose Sodium NF | — | — | 24.0 | — |
| Crospovidone USP | 24.0 | 24.0 | — | 24.0 |
| Part II | | | | |
| Lactose Spray Dried | 54.0 | — | — | 54.0 |
| Microcrystalline Cellulose NF | — | — | 40.0 | — |
| A-Tab (Cal. Phosphate Dibasic | — | 54.0 | — | — |
| Anhyd. USP) | | | | |
| Part III | | | | |
| Magnesium Stearate NF | 6.0 | 6.0 | 6.0 | 6.0 |
| Total | 600.0 | 600.0 | 600.0 | 600.0 |

### Examples 9-12

Method—Direct Compression

Procedure:
1. Mix together the components of part I.
2. Add Magnesium Stearate of Part II to the mix from step 1 and mix to get a final mix.

3. Compress the final mix from step 2 into tablets.

TABLE 3

| | Exam- | Exam- | Exam- | Example |
|---|---|---|---|---|
| | ple 9 | ple 10 | ple 11 | 12 |
| Ingredients | mg/tab | mg/tab | mg/tab | mg/tab |
| Part I | | | | |
| Linezolid | 300.0 | 300.0 | 300.0 | 300.0 |
| Starch 1500 | 100.0 | 100.0 | 100.0 | 100.0 |
| Mannitol | — | — | — | 200.0 |
| Povidone USP (PVP K-30) | 30.0 | — | 30.0 | 30.0 |
| Hydroxy Methyl Cellulose NF | — | 30.0 | — | — |
| Croscarmellose Sodium NF | — | 24.0 | — | — |
| Crospovidone USP | 24.0 | — | 24.0 | 24.0 |
| Lactose Spray Dried | 100.0 | — | — | — |
| Microcrystalline Cellulose NF | 140.0 | 40.0 | 240.0 | 40.0 |
| A-Tab (Cal. Phosphate Dibasic | — | 200.0 | — | — |
| Anhyd. USP) | | | | |
| Part II | | | | |
| Magnesium Stearate NF | 6.0 | 6.0 | 6.0 | 6.0 |
| Total | 700.0 | 700.0 | 700.0 | 700.0 |

### Examples 13-14

Method—Dry Granulation.

Procedure:
1. Mix together the components of Part I and II.
2. Compress the mix from step 1 into tablets (slugs).
3. Mill the tablets from step 2, add components of part III and IV, and mix well to get a final blend.

4. Compress the final blend from step 3 into tablets.

TABLE 4

| | Example 13 | Example 14 |
|---|---|---|
| Ingredients | mg/tab | mg/tab |
| Part I | | |
| Linezolid | 300.0 | 300.0 |
| Starch NF | 100.0 | 100.0 |
| Mannitol | — | 100.0 |
| Povidone USP (PVP K-30) | 30.0 | — |
| Hydroxy Methyl Cellulose NF | — | 16.0 |
| A-Tab (Cal. Phosphate Dibasic Anhyd. USP) | 100.0 | — |
| Crospovidone USP | 24.0 | 24.0 |
| Part II | | |
| Magnesium Stearate NF | 5.0 | 5.0 |
| Part III | | |
| Mannitol | — | 150 |
| A-Tab (Cal. Phosphate Dibasic Anhyd. USP) | 136.0 | — |
| Part IV | | |
| Magnesium Stearate NF | 5.0 | 5.0 |
| Total | 700.0 | 700.0 |

### Examples 15-18

Method—Wet granulation.

Granulation Solution—See table below

Procedure:
1. Mix together the components of part I.
2. Add the granulation solution and mix to form a granulate.
3. Dry wet granulate from step 2 and mill.
4. Add components of part II and III to the milled granulate from step 3 and mix to get a final blend.

5. Compress the final blend from step 4 into tablets.

TABLE 5

| | | Example | Example | |
|---|---|---|---|---|
| | Example 15 | 16 | 17 | Example 18 |
| Ingredients | mg/tab | mg/tab | mg/tab | mg/tab |
| Part I | | | | |
| Linezolid | 300.0 | 300.0 | 300.0 | 300.0 |
| Starch NF | 100.0 | 100.0 | 100.0 | 100.0 |
| Cal. Phosphate Dibasic | 100.0 | 100.0 | 100.0 | — |
| Anhyd. USP | | | | |
| Mannitol | — | — | — | 100.0 |
| Povidone USP (PVP K-30) | 30.0 | 30.0 | 30.0 | 30.0 |
| Hydroxy Methyl Cellulose | — | — | — | — |
| NF | | | | |
| Croscarmellose Sodium NF | — | — | — | — |
| Crospovidone USP | 24.0 | 24.0 | 24.0 | 24.0 |
| Granulation Solution | Ethanol/Purified | iso-propyl | PVP K-30 | PVP K-30 in |
| | Water | alcohol | in Alcohol | Alcohol/Purified |
| | 1:1 | | | Water |
| | | | | 1:1 |
| Part II | | | | |
| Microcrystalline Cellulose | 40.0 | 40.0 | 40.0 | 40.0 |
| NF | | | | |
| Part III | | | | |
| Magnesium Stearate NF | 6.0 | 6.0 | 6.0 | 6.0 |
| Total | 600.0 | 600.0 | 600.0 | 600.0 |

The polymorphic content of each of the pharmaceutical compositions was determined by x-ray powder diffraction (“XRD”). XRD diffractograms were obtained using a Scintag X-Ray powder diffractometer model X'TRA with a Cu tube and a solid state detector. For sampling a round standard aluminum sample holder with a round zero background quartz plate was used. The following scanning parameters were used: Regular scan, i.e., 2-40 degrees 2θ, continuous scan, rate 3.00 degree/min.

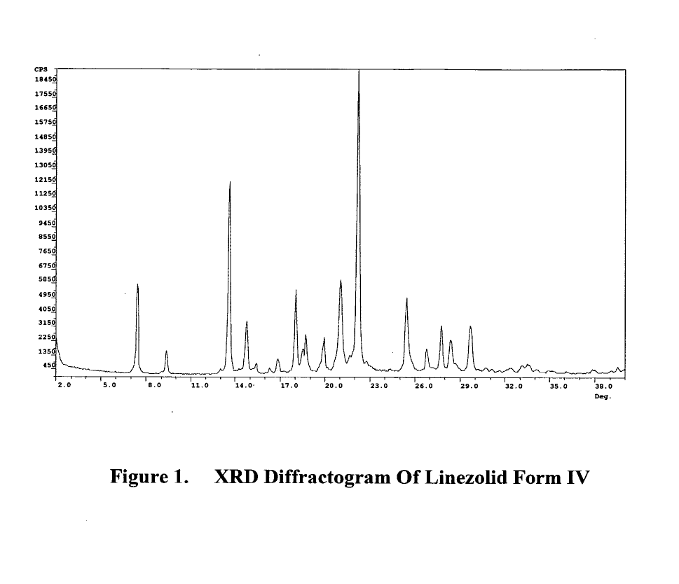

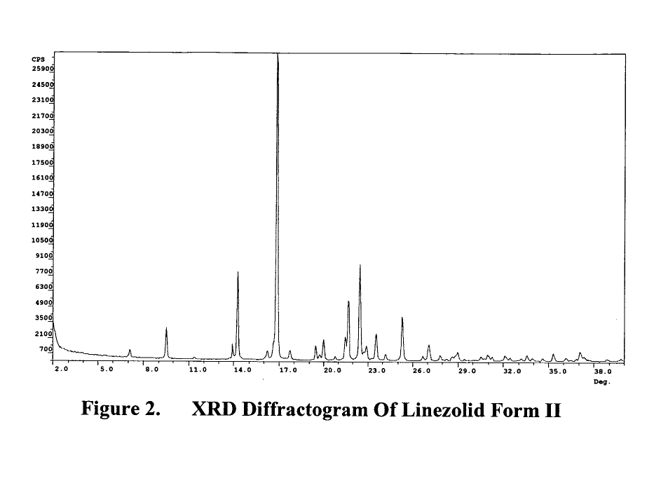

FIG. 1 depicts the XRD diffractogram of linezolid Form IV and FIG. 2 depicts the XRD diffractogram of linezolid Form II.

The characteristic peaks in the XRD diffractogram of linezolid Form II are found at about 7.1±0.2, 9.6±0.2, 14.2±0.2, 16.9±0.2, 21.7±0.2, 22.5±0.2, and 23.6±0.2 degrees 2-theta.

The characteristic peaks in the XRD diffractogram of linezolid Form IV are found at about 7.40±0.2, 9.4±0.2, 13.6±0.2, 14.8±0.2, 15.2±0.2, 15.4±0.2, 16.3±0.2, 16.9±0.2, 18.0±0.2, 18.5±0.2, 18.8±0.2, 21.0±0.2, 22.3±0.2, and 29.7±0.2 degrees 2-theta.

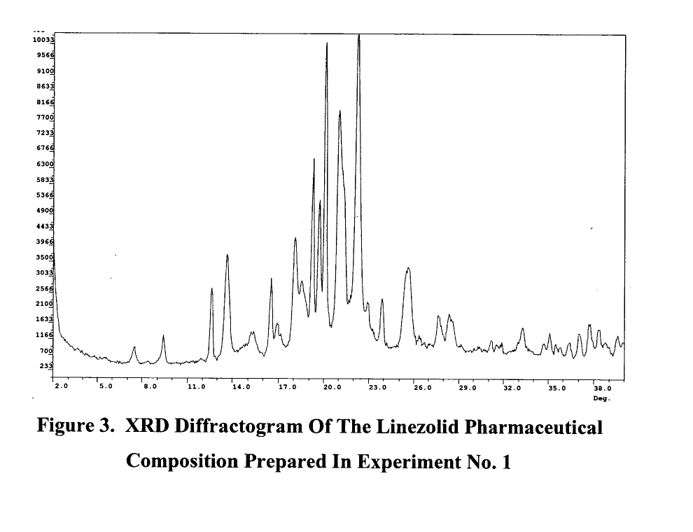

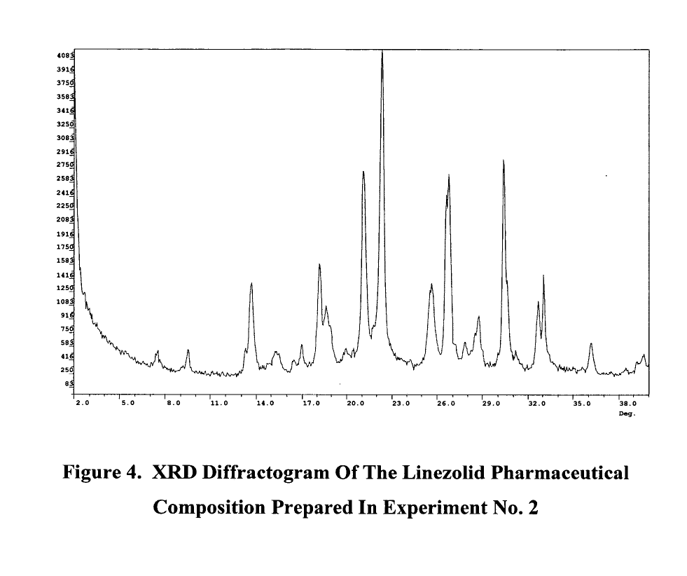

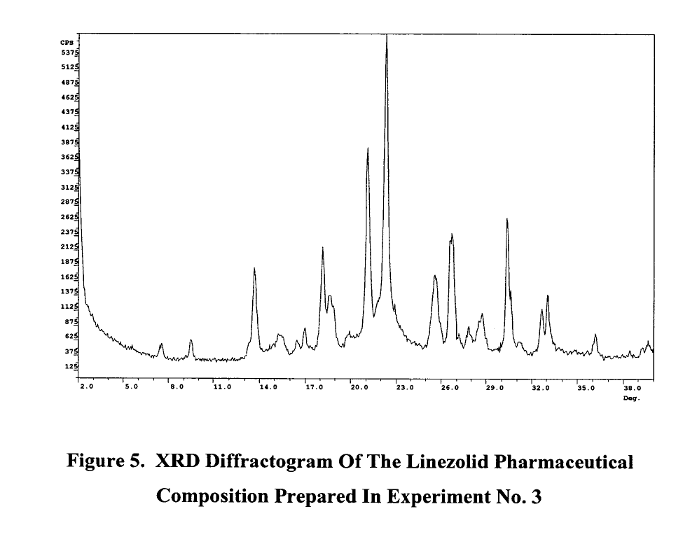

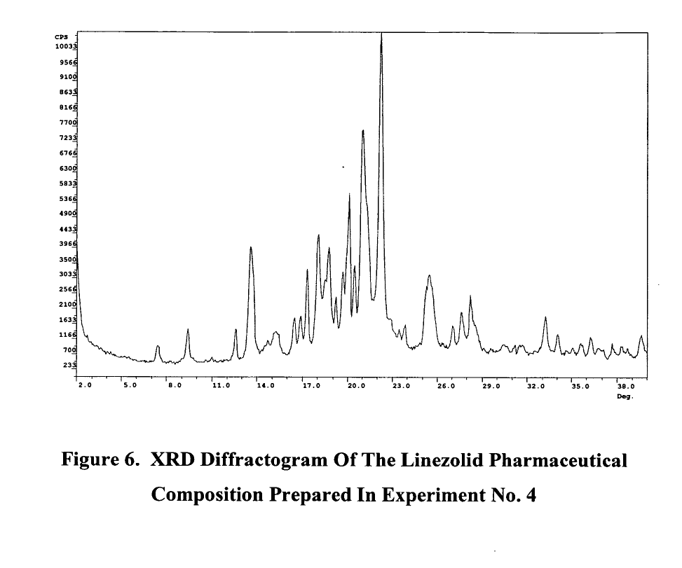

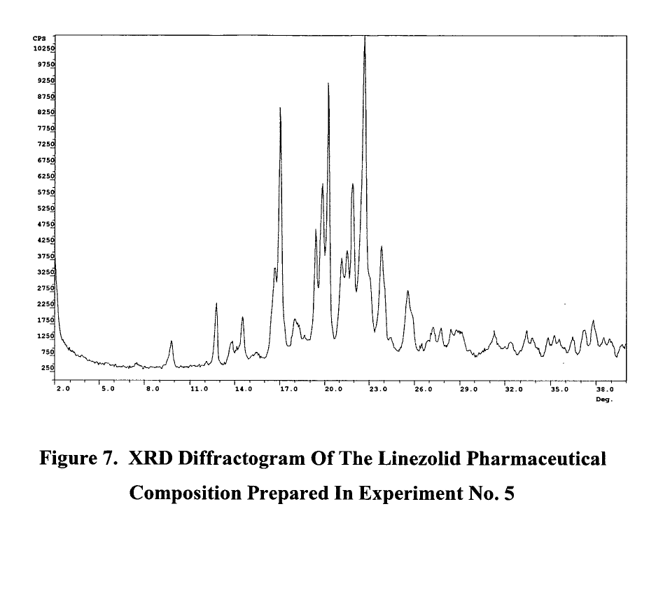

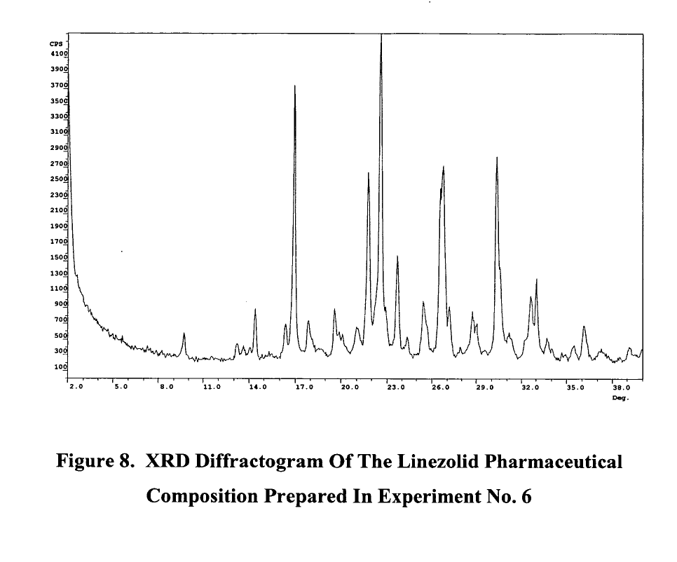

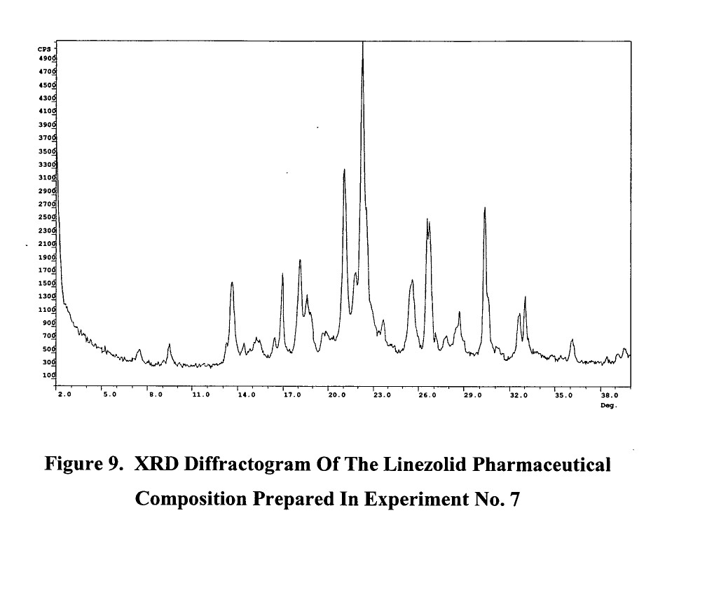

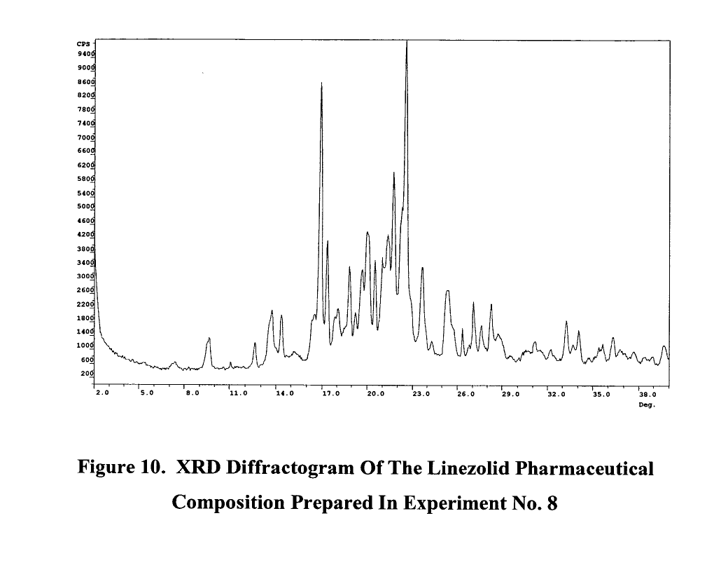

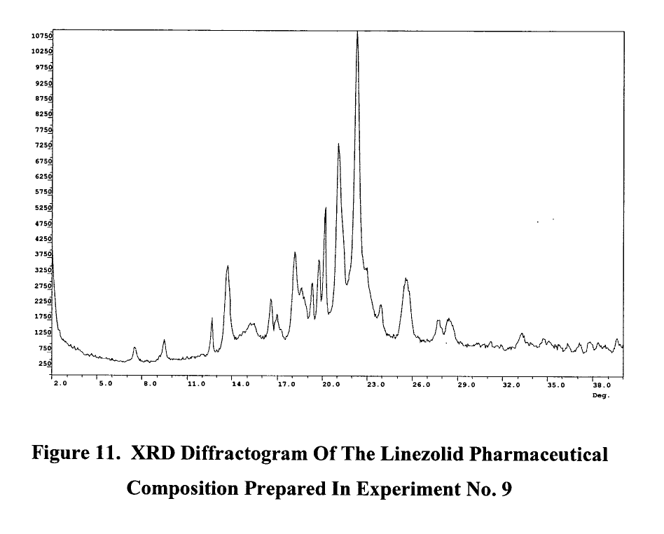

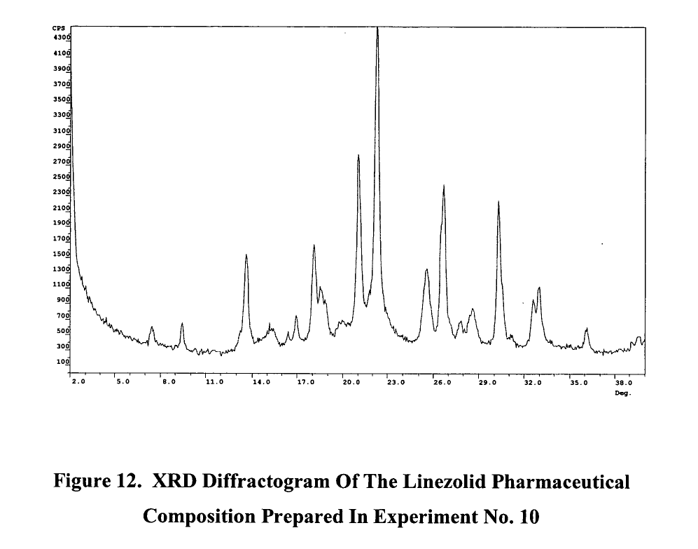

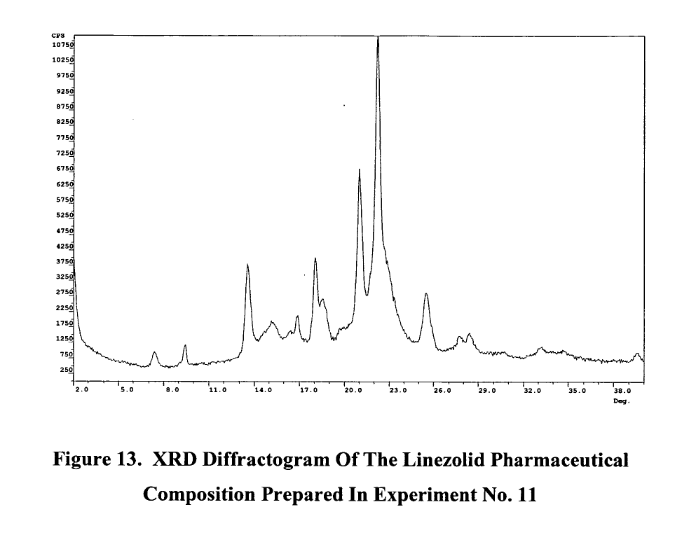

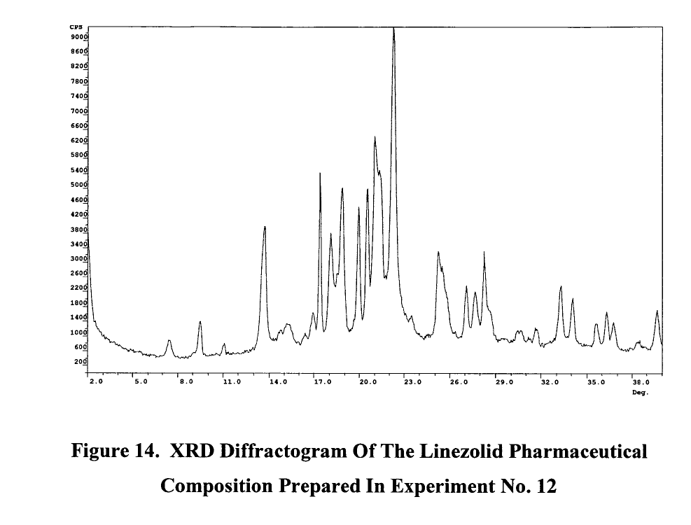

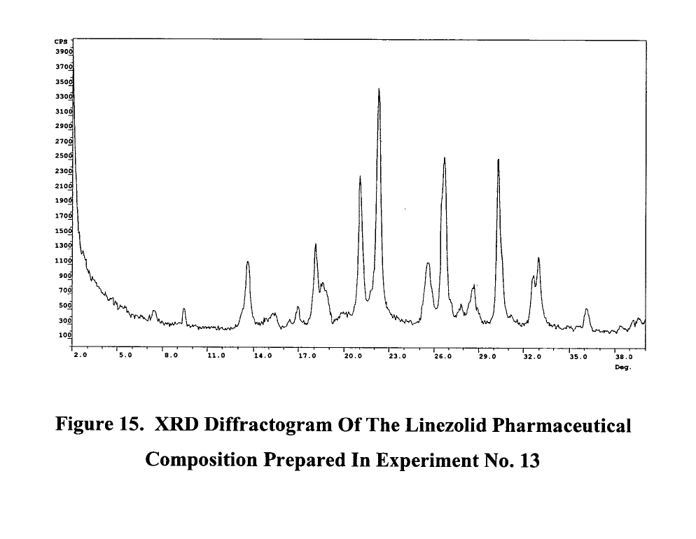

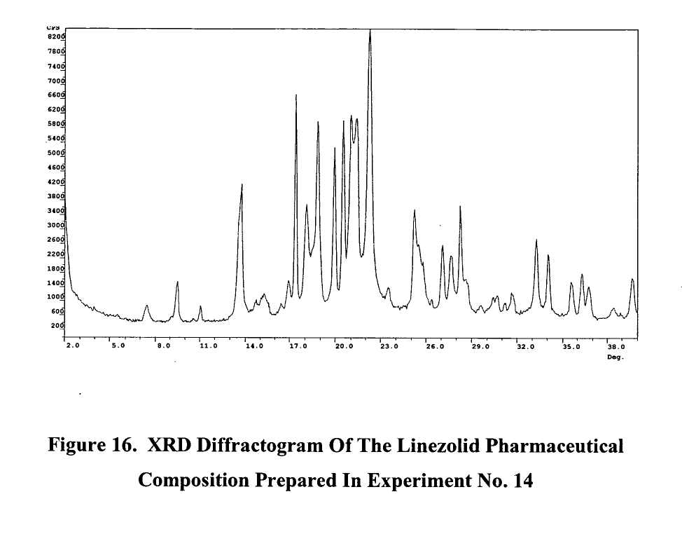

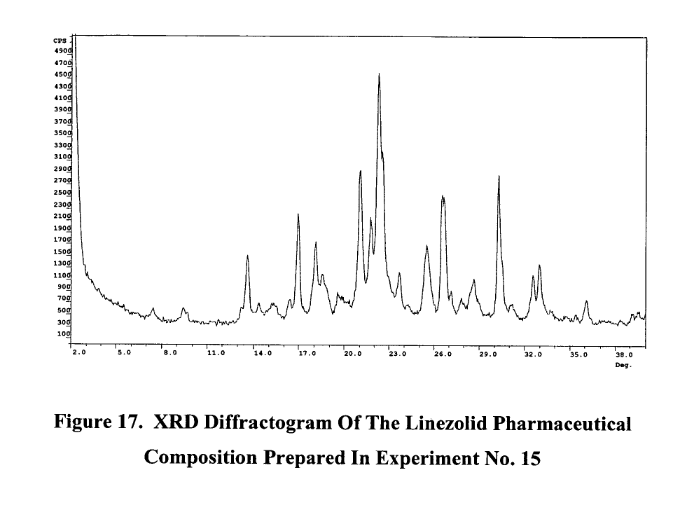

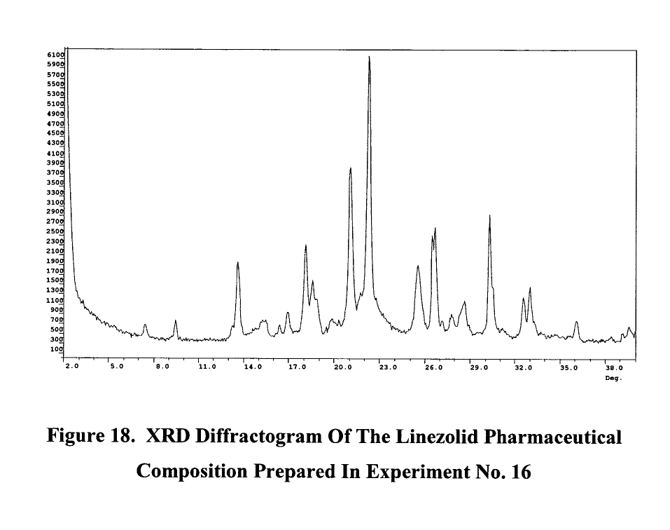

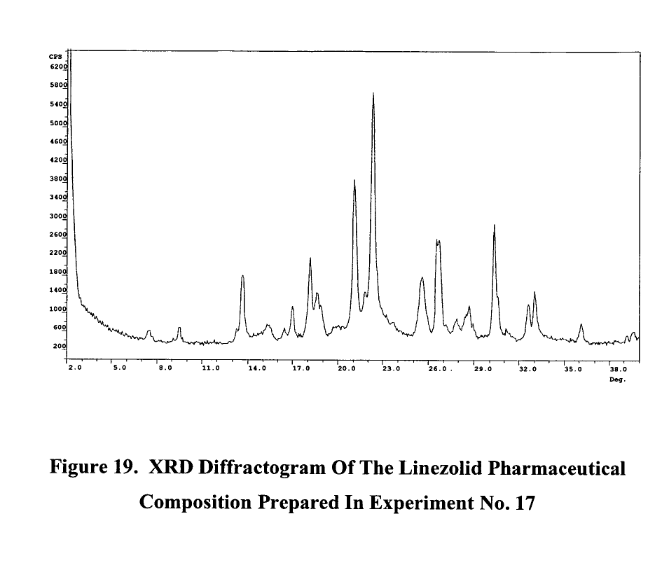

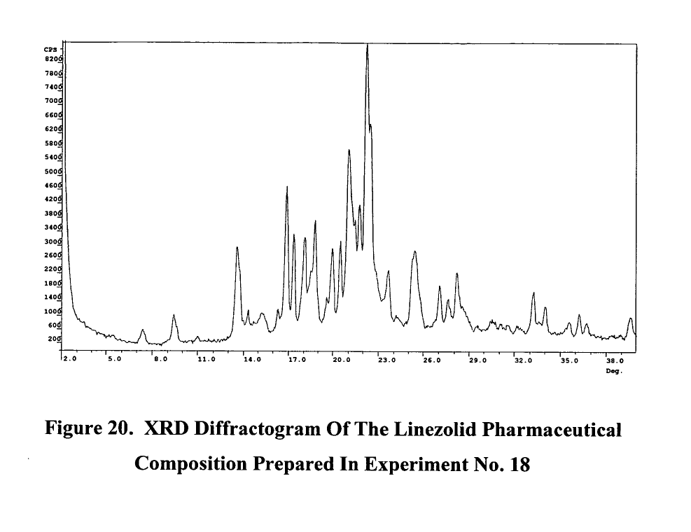

FIGS. 3-20 depict the XRD diffractogram of the pharmaceutical compositions prepared above. By knowing the characteristic peaks in the XRD of linezolid Form II and linezolid Form IV it is possible to determine the crystal Form of the linezolid in each pharmaceutical composition. The XRD diffractogram of the pharmaceutical compositions includes peaks from the linezolid and the excipients included in the pharmaceutical formulation. By knowing which peaks in the XRD diffractogram of the pharmaceutical composition are due to the excipients, it is possible to identify the peaks that are due to linezolid and, therefore, to identify whether the linezolid in the pharmaceutical composition is linezolid Form IV or linezolid Form II. Although, it may not be possible in an XRD diffractogram of a pharmaceutical composition to identify every one of the characteristic peaks of linezolid Form II or linezolid Form IV identified above (for example, because peaks from an excipient may interfere with or overlap with peaks from linezolid), a sufficient number of peaks corresponding to linezolid Form IV or linezolid Form II can be identified for one of ordinary skill in the art to characterize the linezolid in the pharmaceutical composition as linezolid Form IV or linezolid Form II. Typically, the occurrence of peaks in the XRD diffractogram of a pharmaceutical composition at 7.4, 9.4, 13.6, 18.0, 21.0 degrees 2-theta (±0.2) is sufficient to show that the linezolid present in the pharmaceutical composition is linezolid Form IV Similarly, the occurrence of peaks in the XRD diffractogram of a pharmaceutical composition at 14.2, 21.7, 23.6 degrees 2-theta (±0.2) is sufficient to show that the linezolid present in the pharmaceutical composition in addition to form IV is linezolid Form II. The choice of the specific peaks, however, may vary if the excipients used will be different.

Techniques other than X-ray powder diffraction, well known to those of ordinary skill in the art, can also be used to identify and quantify polymorphs in pharmaceutical compositions such as tablets. Representative other methods include, but are not limited to, solid-state NMR and infra-red spectroscopy.

Table 6 summarizes the crystal Form of the linezolid in each of the pharmaceutical compositions upon manufacture.

TABLE 6

| | Granulation Type | | | | | | | | | | | |
|---|---|---|---|---|---|---|---|---|---|---|---|---|
| | | | | | Wet-Ethanol | | | | | | | |
| | Wet-Purified Water | | | | Alcohol 95% | | | | Direct Compression | | | |
| | Example No. | | | | | | | | | | | |
| | 1 | 2 | 3 | 4 | 5 | 6 | 7 | 8 | 9 | 10 | 11 | 12 |
| Crystal | Form | Form | Form | Form | Form | Form | Form | Form | Form IV | Form IV | Form IV | Form |
| Form by | IV | IV | IV | IV | II | II | IV + 40% | II | | | | IV + 15% |
| XRD | | | | | | | Form | | | | | Form |
| | | | | | | | II | | | | | II |

| Granulation Type | |
|---|---|
| Dry | |
| Granulation | Wet Granulation Solution |

Example No.

| | 13 | 14 | 15 | 16 | 17 | 18 |
|---|---|---|---|---|---|---|
| Crystal | Form | Form | Form | Form | Form | Form |
| Form by | IV | IV + 15% | IV + 50% | IV | IV + 10% | IV + 50% |
| XRD | | Form | Form | | Form | Form |
| | | II | II | | II | II |

Table 7 summarizes the peaks of the active ingredient detected in the X-Ray diffractograms of the tablets.

TABLE 7

| Number of | |
|---|---|
| experiment | XRD peaks of the active ingredient detected in the diffractograms of the tablets |
| 1 | Peaks of Form IV at about 7.4, 9.4, 13.6, 18.1, 18.5, 22.2 degrees 2-theta |
| 2 | Peaks of Form IV at about 7.4, 9.4, 13.5, 15.2-15.5 (broad), 16.3, 16.9, 18.0, 18.5, 18.7, |
| | 21.1, 22.3 degrees 2-theta |
| 3 | Peaks of Form IV at about 7.5, 9.5, 13.6, 15.3-15.5 (broad), 16.4, 17.0, 18.1, 18.6, 18.9 |
| | (shoulder), 21.1, 22.4 degrees 2-theta |
| 4 | Peaks of Form IV at about 7.4, 9.4, 13.6, 14.7, 15.1-15.4 (broad), 16.9, 18.0, 18.5, 21.0, |
| | 22.3 degrees 2-theta |
| 5 | Peaks of Form II at about 9.5, 14.2, 16.9, 22.4, 23.6, 21.6, 25.2 degrees 2-theta |
| 6 | Peaks of Form II at about 9.5, 14.2, 16.2, 16.8, 19.4, 21.7, 22.4, 23.6, 25.3 degrees 2-theta |
| 7 | Peaks of Form IV at about 7.4, 9.4, 13.6, 18.1, 18.5, 18.8 (shoulder), 21.0, 22.3 and peak |
| | of Form II at about 14.2, 23.6 degrees 2-theta. |
| 8 | Peaks of Form II at about 9.6, 14.3, 16.9, 21.8, 22.5, 23.7 degrees 2-theta. |
| 9 | Peaks of Form IV at about 7.5, 9.5, 13.6, 16.9, 18.1, 18.5, 21.0, 22.3 degrees 2-theta |
| 10 | Peaks of Form IV at about 7.5, 9.5, 13.6, 16.4, 16.9, 18.1, 18.5, 18.8, 21.1, 22.3, |
| | 25.5degrees 2-theta |
| 11 | Peaks of Form IV at about 7.4, 9.4, 13.6, 15.1-15.4 (broad), 16.4, 16.9, 18.0, 18.5, 21.0, |
| | 22.2 degrees 2-theta |
| 12 | Peaks of Form IV at about 7.4, 9.5, 13.7, 16.4, 16.9, 18.2, 18.8, 25.3, 25.5 degrees 2-theta |
| | and peak of Form II at about 23.6 degrees 2-theta. |
| 13 | Peaks of Form IV at about 7.4, 9.4, 13.6, 15.2-15.5 (broad), 16.9, 18.1, 18.5, 21.0, 22.2 |
| | degrees 2-theta |
| 14 | Peaks of Form IV at about 7.5, 9.5, 14.7, 15.1-15.5 (broad), 16.4, 16.9, 18.1, 18.5, 18.9 |
| | (shoulder), 21.1, 22.3 degrees 2-theta and peak of Form II at about 23.5 degrees 2-theta. |
| 15 | Peaks of Form IV at about 7.4, 9.4, 13.5, 15.2-15.5 (broad), 16.3, 16.9, 18.1, 18.5, 21.1, |
| | 22.2 degrees 2-theta and peak of Form II at about 9.7, 14.4, 23.6 degrees 2-theta. |
| 16 | Peaks of Form IV at about 7.4, 9.4, 13.6, 15.1-15.5 (broad), 16.3, 16.9, 18.1, 18.5, 21.1, |
| | 22.3 degrees 2-theta. |
| 17 | Peaks of Form IV at about 7.5, 9.5, 13.6, 15.2-15.5 (broad), 16.4, 17.0, 18.2, 18.6, 21.1, |
| | 22.3 degrees 2-theta and peak of Form II at about 23.6 degrees 2-theta. |
| 18 | Peaks of Form IV at about 7.3, 9.4, 13.5, 15.3-15.5 (broad), 18.0, 18.5, 21.0, 22.2 degrees |
| | 2-theta and peak of Form II at about 14.2, 21.7, 23.4 degrees 2-theta. |

All the formulations, disregarding the polymorphic composition, were extremely stable at 25° C./60% RH for 6 or 3 months, in the sense that NMT 10% conversion of Form IV to Form II occurred. (See Table 8).

TABLE 8
Stability results of Form IV formulations at 25° C./60% RH

| | Example | Example | Example | Example |
|---|---|---|---|---|
| Interval | No. 1 | No. 2 | No 3 | No. 11 |
| T = 0 | IV | IV | IV | IV |
| T = 3Month | IV | IV | IV | IV |
| T = 6Months | IV | IV | IV | IV |

| | Example | Example | Example | Example | Example | Example | Example | Example | Example | Example | Example |
|---|---|---|---|---|---|---|---|---|---|---|---|
| Interval | No. 4 | No. 7 | No. 9 | No. 10 | No. 12 | No. 13 | No. 14 | No. 15 | No. 16 | No. 17 | No. 18 |
| T = 0 | IV | IV + 40% | IV | IV | IV + 15% | IV | IV + 15% | IV + 50% | IV | IV + 10% | IV + 50% |
| | | II | | | II | | II | II | | II | II |
| T = 3Months | IV + 10% | IV + 40% | IV | IV | IV + 15% II | IV | IV + 15% | IV + 60% | IV | IV + 20% II | IV + 60% |
| | II | II | | | | | II | II | | | II |

It was observed that all the solid compositions prepared according to the methods of the invention by wet granulating with water, isopropanol, or ethanol in the presence of povidone are stable at 40° C./75%RH (See tables 9 and 10), according to the following criteria:

Not more than 30% conversion of linezolid Form IV to linezolid Form II after 3 months (ex. 4, 7, 9, 11, 15, 17, 18);

Not more than 25% conversion of linezolid Form IV to linezolid Form II after 2 months (ex. 4, 7, 9, 11, 12, 15, 17, 18);

Not more than 20% conversion of linezolid Form IV to linezolid Form II after 1 month (ex., 7, 9, 10, 11, 15, 17, 18).

TABLE 9
Stability results of Form IV formulations at 40° C./75% RH (no
detectable conversion to Form II)

| | Example | Example | Example | Example |
|---|---|---|---|---|
| Interval | No. 1 | No. 2 | No. 3 | No. 16 |
| T = 0 | IV | IV | IV | IV |
| T = 1 Month | IV | IV | IV | IV |
| T = 2 Months | IV | IV | IV | IV |
| T = 3 Months | IV | IV | IV | IV |

The results in Table 9 show that there is no detectable conversion of Form VI to Form II in the compositions of Examples 1, 2, 3, and 16.

TABLE 10
Stability results of Form IV formulations at 40° C./75% RH

| | Example | Example | Example. | Example | Example | Example | Example | Example | Example | Example |
|---|---|---|---|---|---|---|---|---|---|---|
| Interval | No. 4 | No. 7 | No. 9 | No. 10 | No. 11 | No. 12 | No. 13 | No. 15 | No. 17 | No. 18 |
| T = 0 | IV | IV + 40% II | IV | IV | IV | IV + 15% II | IV | IV + 50% II | IV + 10% II | IV + 60% II |
| T = 1Month | IV | IV + 50% II | IV | IV | IV | IV + 15% II | IV + 50% II | IV + 60% II | IV + 20% II | IV + 60% II |
| T = 2Month | IV + | IV + 55% II | IV + | IV + | IV + 10% II | IV + 40% II | IV + 70% II | IV + 60% II | IV + 25% II | IV + 60% II |
| | 25% II | | 20% II | 40% II | | | | | | |
| T = 3Month | IV + | IV + 60% II | IV + | IV + | IV + 10% II | IV + 50% II | IV + 80% II | IV + 70% II | IV + 35% II | IV + 65% II |
| | 30% II | | 35% II | 65% II | | | | | | |

It is believed that in the formulations of wet granulation with water or isopropanol, form IV is preserved with time disregarding the excipients present.(Example No. 1, 2, 3, 16)

It is also believed that the formulations of Example Number 9, 11, 12, 13, 15, 17, 18, i.e., where the formulation is dry or other solvents were used instead of or in combination with water, the presence of (povidone (“PVP”) enhances the stability toward polymorphic transformation.

The results of the above described experiments demonstrate the following:

Wet granulation with water or isopropanol is better than granulation with ethanol to provide a formulation with Form IV substantially free of Form II.

Wet granulation with ethanol containing povidone is better than granulation with ethanol in the absence of povidone to provide a formulation with Form IV substantially free of Form II.

Povidone inhibits the conversion of Form IV to Form II even under conditions were the conversion of Form IV to Form II is likely (e.g., in the presence of ethanol). Formulating a pharmaceutical containing povidone by adding the povidone to the composition as a solution of povidone is better than admixing solid povidone with other excipients.

Wet granulation advantageously provides a formulation with Form IV substantially free of Form II.

Dry granulation advantageously provides a formulation with Form IV substantially free of Form II.

It is advantageous to avoid using sugar alcohols, in particular mannitol, as an excipient. Even in dry granulation methods it is advantageous to avoid using sugar alcohols, in particular mannitol, as an excipient.

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited, the entire disclosure of which are incorporated herein by reference.

What is claimed:

1. A pharmaceutical composition comprising polymorphically stable linezolid Form IV and a pharmaceutically acceptable excipient.

2. The pharmaceutical composition of claim 1, wherein the excipient is povidone.

3. The pharmaceutical composition of claim 1, wherein the pharmaceutical composition is substantially free of mannitol.

4. The pharmaceutical composition of claim 1, wherein the pharmaceutical composition is a solid.

5. The pharmaceutical composition of claim 4, wherein the pharmaceutical composition is in the form of a tablet or a capsule.

6. The pharmaceutical composition of claim 1, wherein the pharmaceutical composition shows less than 30 percent conversion of linezolid Form IV to linezolid Form II when the pharmaceutical composition is maintained at 40° C. and 75% relative humidity for 3 months.

7. The pharmaceutical composition of claim 1, wherein the pharmaceutical composition shows less than 25 percent conversion of linezolid Form IV to linezolid Form II when the pharmaceutical composition is maintained at 40° C. and 75% relative humidity for 2 months.

8. The pharmaceutical composition of claim 1, wherein the pharmaceutical composition shows less than 20 percent conversion of linezolid Form IV to linezolid Form II when the pharmaceutical composition is maintained at 40° C. and 75% relative humidity for 1 months.

9. A pharmaceutical composition comprising polymorphically pure linezolid Form IV and a pharmaceutically acceptable excipient.

10. The pharmaceutical composition of claim 9, wherein the linezolid Form IV is polymorphically stable linezolid Form IV.

11. A pharmaceutical composition comprising linezolid Form IV substantially free of linezolid Form II and a pharmaceutically acceptable excipient.

12. The pharmaceutical composition of claim 11, wherein the linezolid Form IV is polymorphically stable linezolid Form IV.

13. A pharmaceutical composition comprising linezolid Form IV and a pharmaceutically acceptable excipient, wherein the pharmaceutically acceptable excipient is an excipient that limits the conversion of linezolid Form IV to Form II.

14. The pharmaceutical composition of claim 13, wherein the pharmaceutical composition is substantially free of linezolid Form II.

15. The pharmaceutical composition of claim 13, wherein the excipient is povidone.

16. The pharmaceutical composition of claim 13, wherein the pharmaceutical composition is substantially free of mannitol.

17. A method of manufacturing a pharmaceutical composition comprising linezolid Form IV comprising wet granulating linezolid Form IV with a pharmaceutically acceptable excipient using a granulating solvent that is substantially free of ethanol.

18. The method of claim 17, wherein the linezolid Form IV is substantially free of linezolid Form II.

19. The method of claim 17, wherein the granulating solvent selected from the group consisting of water and isopropanol.

20. The method of claim 17, wherein the pharmaceutically acceptable excipient comprises povidone.

21. A method of manufacturing a pharmaceutical composition comprising linezolid Form IV comprising wet granulating linezolid Form IV with a pharmaceutically acceptable excipient using a granulating solvent selected from the group consisting of water, isopropanol, or ethanol in the presence of povidone.

22. The method of claim 21, wherein the linezolid Form IV is substantially free of linezolid Form II.

23. The method of claim 21, wherein the granulating solvent comprises water substantially free of ethanol.

24. The method of claim 21, wherein the granulating solvent comprises isopropanol substantially free of ethanol.

25. The method of claim 21, wherein the granulating solvent comprises ethanol in the presence of povidone.

26. A method of manufacturing a pharmaceutical composition comprising linezolid Form IV comprising dry granulating linezolid form IV with a pharmaceutically acceptable excipient.

27. The method of claim 26, wherein the linezolid Form IV is substantially free of linezolid Form II.

28. The method of claim 26, wherein the pharmaceutically acceptable excipient comprises povidone.

29. A method of manufacturing a pharmaceutical composition comprising linezolid Form IV comprising admixing linezolid form IV with a pharmaceutically acceptable excipient to provide a mixture and direct compressing the mixture.

30. The method of claim 29, wherein the linezolid Form IV is substantially free of linezolid Form II.

31. The method of claim 29, wherein the pharmaceutically acceptable excipient comprises povidone.

32. A method of limiting the conversion of linezolid Form IV to linezolid Form II during formulation of a pharmaceutical composition comprising linezolid Form IV and a pharmaceutically acceptable excipient or of limiting the conversion of linezolid Form IV to linezolid Form II in the pharmaceutical composition comprising at least one of:
i. formulating the pharmaceutical composition by wet granulation using a granulating solvent that is substantially free of ethanol;
ii. formulating the pharmaceutical composition by wet granulation using a granulating solvent, wherein during formulation the granulating solvent contacts the linezolid Form IV in the presence of povidone;
iii. including povidone in the pharmaceutical composition; and
iv. keeping the pharmaceutical composition substantially free of sugar alcohols.

33. The method of claim 32, wherein the method comprises formulating the pharmaceutical composition by wet granulation using a granulating solvent that is substantially free of ethanol and the granulation solvent is selected from the group consisting of water and isopropanol.

34. The method of claim 32, wherein the method comprises keeping the pharmaceutical composition substantially free of sugar alcohols and the sugar alcohol is mannitol.

35. The method of claim 17, wherein the method comprises keeping the pharmaceutical composition substantially free of sugar alcohols and the sugar alcohol is mannitol.

36. The method of claim 26, wherein the method comprises keeping the pharmaceutical composition substantially free of sugar alcohols and the sugar alcohol is mannitol.

37. The method of claim 29, wherein the method comprises keeping the pharmaceutical composition substantially free of sugar alcohols and the sugar alcohol is mannitol.
